(19) United States
(12) Patent Application Publication
(10) Pub. No.: US 2024/0282437 A1
(43) Pub. Date: Aug. 22, 2024

(54) PROCEDURAL AND SURGICAL INFORMATION ALERT SYSTEM

(71) Applicant: Alcon Inc., Fribourg (CH)

(72) Inventors: Gregory A. Eippert, Concord, OH (US); James Bowens, Mentor, OH (US)

(21) Appl. No.: 18/652,525

(22) Filed: May 1, 2024

Related U.S. Application Data
(63) Continuation of application No. 17/348,722, filed on Jun. 15, 2021.
(60) Provisional application No. 63/039,310, filed on Jun. 15, 2020.

(51) Int. Cl. G16H 40/20; A61B 34/00

(52) U.S. Cl. CPC G16H 40/20; A61B 34/25; A61B 2034/256

(57) ABSTRACT

The procedure and surgical information alert system 10 is a surgical management system for capturing, storing, processing, tracking, and sharing patient and procedural identification data, including implant data, during medical/surgical procedures performed at multiple medical offices 20 and/or surgical center 30 locations. The procedure and surgical information alert system 10 allows for accurate verification of the predetermined procedure verified between the physician and patient during the scheduling process at a medical office 20, which is confirmed at that time by the patient's signature. On the day of the surgical procedure, the patient information is re-verified by the patient at a surgery center. Prior to the surgical procedure, a time-out alert warning 240 is generated to freeze use of the system until physician verification of the patient information is confirmed. Implant inventory alert warnings 192 are provided to authorized surgery center staff based on predictive implant inventory needs.

### CROSS-REFERENCE TO RELATED APPLICATIONS

This application claims the benefit of provisional U.S. patent application Ser. No. 63/039,310 filed on Jun. 15, 2020, the entire contents of which is incorporated herein by reference.

### FIELD OF INVENTION

The present application provides an improved procedure and surgical information planning and alert system generally, and more particularly, to an improved method and apparatus for providing patient confirmation, surgical details and surgery center management information for multiple surgery centers, including alerts regarding physician and patient confirmation, procedures and inventory regarding surgical and surgery center management.

BACKGROUND

From 1995 to 2000, The Joint Commission, which reviews health care organizations to improve standards and issue accreditation evaluations, determined there was a 13.5% occurrence of wrong site surgeries in cases they had reviewed. Wrong site surgery is surgery performed on the wrong side or wrong site of the body. Wrong procedure surgery generally references instances where the wrong surgical procedure is performed. Wrong person surgery is an instance where surgery is performed is on the wrong patient.

Wrong site surgery and even wrong patient surgery are a constant concern for surgeons or physicians performing outpatient or inpatient procedures. The Joint Commission projected as many as 50 wrong-site incidents occur each week in the United States. Such surgeries can be devastating for the patient and have a negative impact on the doctor, doctor's practice and surgical team. Fortunately, wrong site surgeries are preventable medical errors.

Wrong site surgeries are believed to occur due to a collection of small errors which may arise during various instances of patient processing or as a result of other factors, including: booking, verification, distractions, inconsistent site marking and time-out errors. To combat these errors, The Joint Commission has proposed a Universal Protocol Initiative of Standardized Preoperative Preparations, which should include 3 elements: Patient/Procedure Identification; Site Marking; and a Time-Out procedure.

In ophthalmic surgery procedures, for example, there are many choices and factors to be verified to prevent a wrong site surgery or a wrong procedure. Additionally, managing the inventory of intraocular lenses (IOL's) across multiple surgery centers to ensure the desired IOL's that might possibly be used by a particular surgeon during a particular surgery are available on demand, also requires consideration of numerous factors and choices. While a surgeon may plan the particular type and power of the lens to be used for a patient, changes are often made during surgery, so a range of IOL products are required to be available. For example, if a 20 diopter lenses is planned, a range of 10 to 30 diopters is desired to be available in the event a change becomes necessary during the surgical procedure. A typical surgery center inventory of IOL's might include as many as 1,600 IOL's of various brands, types and powers. Since such IOL implants are not paid for until they are used, the IOL vendor/manufacturer is also interested in determining surgery center inventory information. The numerous choices include:
1. Lens Options (Standard , Toric, Multifocal)
2. Right or Left eye
3. Near or Distance vision
4. Laser option for Lens Power calculation
5. Laser option for Astigmatism management
6. Medication delivery option

While various prior art systems have presented aspects of inventory management, for example, U.S. Pat. No. 10,748,115, such systems to do not interface across all aspects of a patient procedure and surgical information planning, or with processing patient information at the surgery centers or providing alert warnings. To avoid wrong site, wrong procedure and wrong patient surgery problems, as well as patient recovery, and improved implant inventory management over multiple surgery centers, a new and more reliable system is needed.

### SUMMARY OF INVENTION

The procedure and surgical information alert system is a surgical management system for capturing, storing, processing, tracking, and sharing patient and procedural identification data, including implant data, during medical/surgical procedures performed at multiple medical offices and/or surgical center locations. The procedure and surgical information alert system allows for accurate verification of the predetermined procedure verified between the physician and patient during the scheduling process at a medical office, which is confirmed at that time by the patient's signature or other form of identifying feature or information, such as a finger print. On the day of the surgical procedure, the patient information is re-verified by the patient, perhaps at an alternate surgery center location different from the initial medical office. The re-verification may be in a similar manner as the initial verification, and the information is preferably presented on display devices or monitors for continuous verification through the medical and surgical visits for reference both before, during and after the surgical procedure.

Initially the system is used to schedule the medical or surgical procedure with the patient while the patient is with the medical care provider. The system includes an input device having a microprocessor, a bus connector, an input data receiver, an internet connection or cellular connection, a memory storage area and an application program. To accomplish input of the initial data, the input device may include any information gathering device, such as either a desk top computer located in the room, or a portable information gathering device (i.c., an electronic tablet or a cellular telephone), for collecting patient and procedural identification data. When a patient is preparing and planning a surgery with the doctor, this system and input device will be used to input and verify a patient's identifying criteria including but not limited to full name, date of birth, procedure to be performed and any specifics about the procedure, including the anticipated device(s) to be implanted. This initial data input or information will be collected and stored within the system using the input device, presented on the information gathering device, and electronically presented to the patient to review.

After reviewing the information, the patient is asked to verify that the information is correct by electronically signing on the information gathering device, using one of several possible formats provided to the input device, and a time stamp is automatically stored to document the input. For example, the patient may confirm by alternatively having their fingerprint scanned by placing their finger on or inside a fingerprint reader. Any type of verification may be used, which enables the system to properly identify the patient and verify the procedure and patient information, such as alternative biometric identification system or conventional electronic patient identification systems, such as coded bracelets and scanning devices. This electronic information will then be transmitted via the system components for storage in the memory storage area, which may be either a central on-site server or a remote cloud- based secure website. Once the patient and procedural identification data is stored, the application program enables later data access at the surgical center or other location where the patient is having their procedure or surgery.

On the day of the scheduled procedure or surgery, upon arrival to a surgical center, the system is again used to input patient identification information, the time of arrival is documented, and surgical and procedural information stored within the system are retrieved for review by the patient. The patient's initial signature may appear on an input device or screen of an information gathering device to verify initial acceptance of the surgical plan that was prepared in the physician's office. If any specifics of the procedure have been changed since initial verification due to a patient's request, this is clearly identified to the receptionist and these changes are verified with the patient before the second verification. Once re-confirmed as accurate by the patient, this information will then be re-verified by the patient, with the patient electronically signing or providing finger print confirmation, similar to the manner it was originally confirmed in the physician's office. In the event the information is incorrect, the medical care provider and patient discuss and confirm any changes or corrections to the procedure.

Prior to the day of the scheduled procedure or surgery, it is understood that the system provides medical staff with an alert or report, which may be automatically generated, regarding each specific surgical date to ensure that the necessary inventory of all implants, supplies and equipment for all patient surgeries are on hand and in place at the desired surgery center location. Lead times of as much as 2-4 weeks prior to any surgery requiring an implant, ensures that medical staff avoid rushing immediately prior to any surgery date to fill inventory requirements.

Implant vendors may likewise be automatically or manually provided with an alert notification of inventory requirements, enabling any necessary ordering to be done well in advance, and an optional online ordering link provided for manual inventory ordering by medical staff. Also, surgery schedules are able to be generated for individuals or groups of surgeons for a given date, with surgical details provided for cach patient.

In addition to the written description provided to summarize the procedure to be performed, an image or partial image of a human form is also provided, with the proper body part where the procedure is to be performed highlighted, for example by circling, surrounding or colored in a bright color, such as red, or otherwise drawing attention to the correct surgery location. By highlighting the surgery location on a human image, the patient and medical staff are more readily able to visually observe and confirm the proper surgery site, and thereby avoid a wrong site surgery. In the event the re-signing or electronic re-verification procedure is not completed, the surgery procedure may not proceed.

After the patient's information data and surgical procedure are electronically re-verified and auto-time stamped, an identification bracelet (ID bracelet) including a programmed ‘chip’ or a commercially available RFID chip is placed on the patient's ID bracelet for electronically connecting with the system, and for retrieval from the system at various stages and locations during the patient's surgical experience. Again, it should be understood that any appropriate biometric verification device may also be used at this time to verify the patient's identity and authorization to proceed, such as a finger print scan on another portable information gathering device.

Upon arrival to the pre-operative area of the surgery center, the patient's ID bracelet will be electronically scanned, using appropriate hard wired or WiFi interconnected scanning equipment, and information regarding the patient and the surgical procedure, as well as a time stamp, will be transmitted and stored within the system, and the data viewed on another tablet or information gathering device (or bed monitor) electronically interfacing with the system, and provided to travel with the patient, for example, at the foot of the patient's bed. The advantages of multiple information confirmations at multiple medical or surgery center locations, enables reduced risk of erroneous procedures. An additional advantage of the system enables data gathering concerning the time spent by medical staff with cach patient within cach stage of the procedure and at each location. The storage of such historical data within the memory storage area of the system enables the medical staff and surgical center locations to compare efficiencies for each team, for each procedure at each location. Where differences are observed, such as insufficient or excessive time spent, by particular medical professionals at particular locations, the medical staff may be made aware of the differences, and an investigation and any necessary corrections may be made. The use of accurate historical data across multiple medical and surgical center locations is enabled by the use of an atomic clock within the system. Atomic clock reporting of historical data provides the medical staff and administration with performance analysis information not previously available.

Information such as time in the OR, start time of the procedure, end time of the procedure and time exiting the surgical suite are all captured and can be automatically entered into the patient's record. Additionally, or alternatively, beacon technology, or a software positioning system, may be incorporated into the system. Using beacon devices positioned throughout a surgery center and associated software, the application program automatically monitors and time stamps movement of the patient through their surgical experience via electronic detection (for example, Bluetooth detection) of the patient's ID bracelet, and/or bed monitor device, or other Smart device, as the patient is transported past beacon devices located at various locations throughout the surgery centers.

Upon transfer into the surgical suite or operating room, the patient's ID bracelet may again be scanned and the patient's information presented on another operating room computer or large “heads up” monitor visible throughout the entire surgical suite. By presenting the patient's information, including the visual image of a human form with the proper surgery site highlighted, the system allows for precise patient and procedure confirmation to the surgical staff and physician. The information shown will again include the patient's identifying criteria including but not limited to full name, date of birth, the specific procedure to be performed, written and visual descriptions of whether the site is on the right or left side of the body, and what special considerations were discussed.

Prior to the surgery or procedure, the time-out procedure is performed in the operating room immediately before the planned procedure is initiated. The time-out procedure is the last component of the Universal Protocol suggested by The Joint Commission, and represents the final review and reassurance of accurate patient identity, surgical site and planned procedure. A time-out alert warning, preferably provided in the color red, is provided on the operating room computer or monitor of the system. The display of the red time-out alert warning effectively freezes or pauses the system, and the alert warning may not be removed until the system receives proper confirmation of performance of the physician's time-out review. Activation by the physician of an electronic confirmation “button” on the input device is required to confirm the time-out procedure has been performed, and permit removal of the time- out alert warning and continued viewing and use of the system. Where a voice activation control is provided within the application program of the system, the physician confirmation may be provided by a voice command or by manual confirmation. The internal atomic clock also documents the exact time in which the time-out procedure was performed for later historical data analysis.

After the time-out procedure is conducted and the time-out confirmation button is activated, the time-out alert warning is removed, system function resumes, and the system application program stores an indication of the doctor and the time the time-out procedure was indicated as performed. In the event the time-out procedure cannot be properly confirmed, the surgery is postponed until there is proper confirmation. Once the surgery or procedure is completed, the patient information data is stored for future reference.

It is noted that included in the patient's information data to be verified during the time-out procedure may include additional information placed by the physician that will remind the surgeon of specific technical data that may be necessary for the procedure. This could include but not be limited to the power of the intraocular lens to be used in cataract surgery, the make or model of a specific prosthetic device to be used or even anticipated anxieties that the patient may encounter.

Following the surgery or procedure, the patient is transported to the post-operative area of the surgery center for recovery prior to discharge. The tablet monitor or information gathering device provided at the foot of the patient's bed continues to travel with the patient, and is used to document departure and arrival times at each location, either by scanning of the patient's ID bracelet or a beacon system used within the surgery center. Prior to discharge, the patient's ID bracelet with the RFID chip is permanently erased. Post surgery, the system again provides medical staff with an alert or

report, which may be automatically generated, regarding an updated inventory of all implants, supplies and equipment remaining on hand and in place at the desired surgery center location. Implant vendors may likewise be automatically provided with or manually obtain an alert notification of inventory usage and future requirements.

The present system provides a method or process which is intended to avoid wrong site surgery errors, and to provide improved efficiencies in various aspects of the medical procedures performed, including timing, equipment, set-up, accurate time data records for the surgery center and inventory control across multiple medical and/or surgical centers. The system may be suitably implemented as a computer based system using a number of computer devices having microprocessors, and where the application program comprises programming instructions, that when executed by the microprocessors, will cause the microprocessor to automatically store or extract data from messages transmitted via the bus, to automatically determine the specific procedural and surgical information for the individual patient, provide a time- out alert warning which cannot be removed prior to performance of the surgical procedure without proper physician confirmation. Such computer software instructions are stored in a suitable non-transitory manner and may reside on computer readable media, such as solid state storage devices, such as RAM, ROM or the like memory, magnetic storage devices, such as CD-ROM, CD-RW, DVD, Blue Ray Disc or the like, remote computer or cloud-based storage, or as methods implemented by such systems and software. The system may be implemented on HIPAA-compliant computing devices, workstations, computer servers or mobile devices such as cell phones, tablets, iPads™, iPods™ and the like. It is critical that patient information, surgery, equipment and inventory data be collected and shared, to keep track of where, when and what surgical procedures took place, that alerts are provided to ensure the proper time-out procedure has been performed, and that the necessary levels of inventory are promptly updated to provide alert notifications to medical staff to ensure required equipment and IOL's are available for future procedures.

### BRIEF DESCRIPTION OF THE DRAWINGS

FIG. 1 schematically illustrates a sample main computer screen of the system where the desired patient information and surgical procedure information may be selected for input/entering or output/reporting.

FIG. 2 schematically illustrates a sample computer screen of the system where new patient information may be input/entered.

FIG. 3 schematically illustrates a sample computer screen of the system where a patient's surgical procedure information may be input/entered.

FIG. 4 schematically illustrates a sample computer screen of the system where the patient is asked to verify that their patient and surgical procedure information is correct by electronically signing to provide a time stamp of the verification within the system database.

FIG. 5A illustrates a patient electronically signing the verification page of FIG. 4 within the system.

FIG. 5B illustrates an alternate biometric electronic verification device which may be used by a patient to electronically verify the accuracy of their patient and surgical procedure information, as in FIG. 4.

FIG. 6 illustrates a sample computer screen of the system presented to the patient at the surgery center on the day of the scheduled surgery, and where the surgical and procedural information stored within the system is retrieved for review by the patient, the initial signature is visible, and after confirming the accuracy of the information, the patient is asked to re-verify the information by again electronically signing or providing finger print confirmation.

FIG. 7 schematically illustrates an identification bracelet which is provided for the patient to wear after the patient's information data and surgical procedure are electronically re-verified, which ID bracelet includes an RFID chip for electronically connecting with the system, and retrieving patient information from the system at various stages and locations during the patient's surgical experience, whether by scanning or beacon technology.

FIG. 8 schematically illustrates a portable information gathering device for use in connection with a patient's re-verification of their information on the day of their scheduled procedure, having an electronic finger print identification confirmation, and which device-or bed monitor-may travel with the patient at the foot of the patient's bed during their surgical experience.

FIG. 9 schematically illustrates a sample screen of the system to be presented on a large monitor and/or other computer screen within the operating room (OR) suite as the patient enters the OR for their scheduled procedure, and showing the TIME IN to the OR, as well as the red highlighted TIMEOUT alert warning, which alert warning will continue to be visible and pause further use of the system until the proper physician verification of the scheduled procedure on the patient is received.

FIG. 10 schematically illustrates the sample screen of FIG. 9, but with a time stamp for the TIMEOUT procedure updated as shown, and with the highlighted TIMEOUT alert warning changed to green, indicating the TIMEOUT was completed and the confirmation was entered into the system by the physician, whether by electronically “pushing” a button, or by a voice command to the system.

FIG. 11 schematically illustrates an operating room suite with the large system monitor positioned to be visible throughout the room.

FIG. 12 schematically illustrates the patient information and surgical procedural information system screen showing the human form with the eye which is the subject of the surgical procedure highlighted, and detailed information illustrated on portable information gathering devices or OR suite computer screens.

FIG. 13 schematically illustrates an alternate surgical procedure for which the system may be used, and showing the human form with the designated appendage which is the subject of the surgical procedure highlighted for ease of reference and avoidance of a wrong site surgery.

FIG. 14 schematically illustrates a sample report generated by the system to provide post operative information on a particular surgeon's productivity.

FIG. 15 schematically illustrates a sample pre-operative report generated by the system to provide a particular surgeon and surgery center staff with a schedule of sample patient's and their planned implant inventory information for a particular surgery day.

FIG. 16 schematically illustrates a flow chart of the programmed process and method for the procedure and surgical information alert system of this application.

FIG. 17 illustrates a schematic diagram showing operational aspects and components of the platform for the procedure and surgical information alert system of this application.

FIG. 18 illustrates a sample report generated by the system for a specific physician of one sample daily planned specific IOL's to be used.

FIG. 19 illustrates a sample report generated by the system for a specific physician similar to FIG. 18 but of the daily planned percentages of specific

IOL's to be used.

FIG. 20 illustrates a sample inventory ordering report provided to surgery center staff by the system for a specific surgery day at a specific surgery center, based on the planned IOL usage by the physicians at the surgery center, and the additional predictive inventory needs required to ensure sufficient IOL supplies.

FIG. 21 illustrates a sample report generated by the system and

showing the physicians usage of patient implants and equipment usage at a particular surgery center.

FIG. 22 illustrates a sample report generated by the system and showing patient throughput analytics for each physician in at surgery center.

FIG. 23 illustrates a sample report generated by the system showing patient average throughput cycle times for cach physician in a surgery center.

FIG. 24 is a simplified block diagram schematically illustrating an exemplary embodiment of components of the procedure and surgical information alert system using the computer implemented method according to the present application.

### DETAILED DESCRIPTION FOR CARRYING OUT THE INVENTION

The embodiments of the present system, method and process 10 provide a procedure and surgical information alert system, as well as a surgical management system for capturing, storing, processing, tracking, and sharing patient and procedural identification data, implant inventory data, and physician performance data for medical/surgical procedures performed at multiple medical offices and/or surgical center locations. It is understood that a description has been provided which may be suitably used to implement the procedure and surgical information alert system. In addition to creating, processing and providing architecture that is compliant with the Health Insurance Portability and Accountability Act of 1996 (HIPAA), which enables authorized access to patient health information records, the present system also provides a surgical management processing system that automatically receives physician and surgery center performance data, and equipment and implant usage data via WiFi or cellular network interfaces with information gathering devices and patient RFID bracelets before, during and after planned surgical procedures. A data processing system creates desired patient, physician, medical office, surgery center, equipment and implant inventory records in an electronic database for each medical office and associated surgery centers, and automatically distributes the generated records in response to authorized requests from authorized personnel.

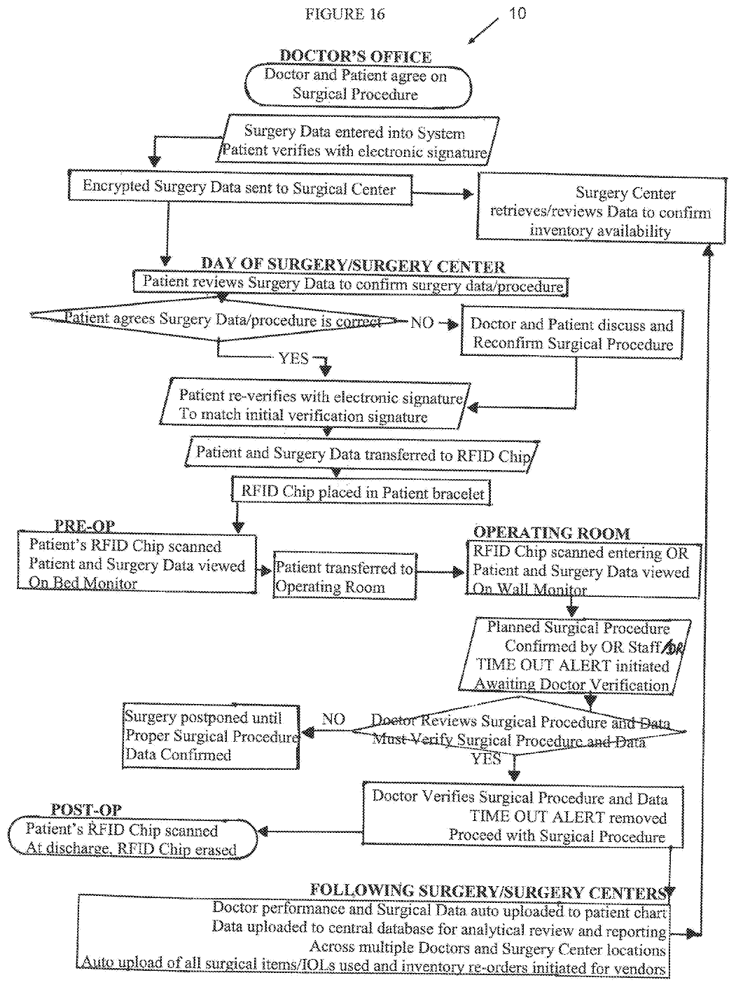

FIG. 16 shows a diagram illustrating, by way of example only, the input, output and decision points provided within the procedure and surgical information alert system. As shown, a time stamp is automatically entered into the surgery center reporting system at each input, output and decision point related to a specific patient, using an atomic or other accurate time keeping clock, internal to the system, to enable later historical data analysis and comparisons of medical staff performance, and various patient procedures and facilities across numerous patients. Additionally, the system records any desired surgical parameters utilized, all signature verifications, and time-out confirmations for each patient. For example, tourniquet times may also be included as illustrated in FIG. 13.

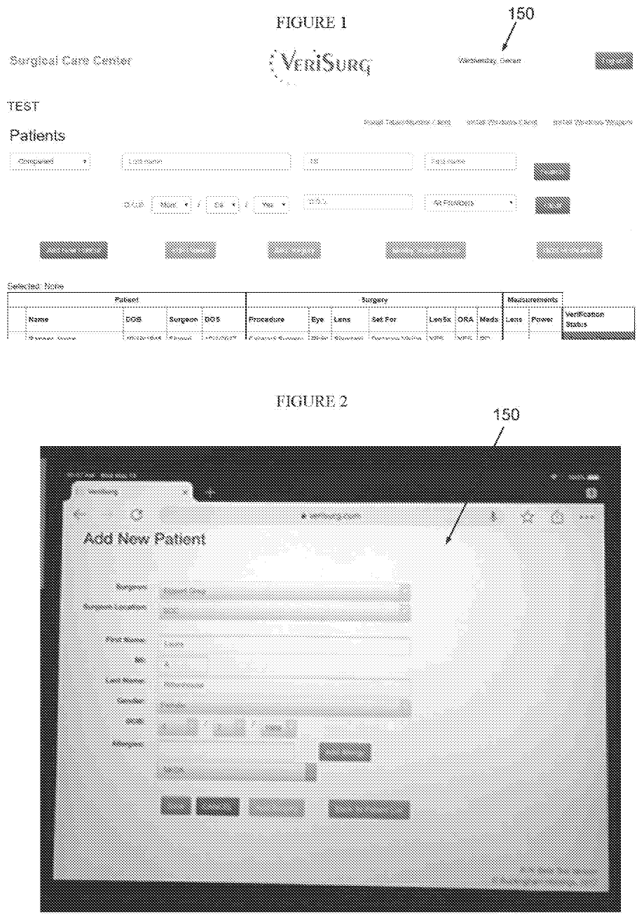

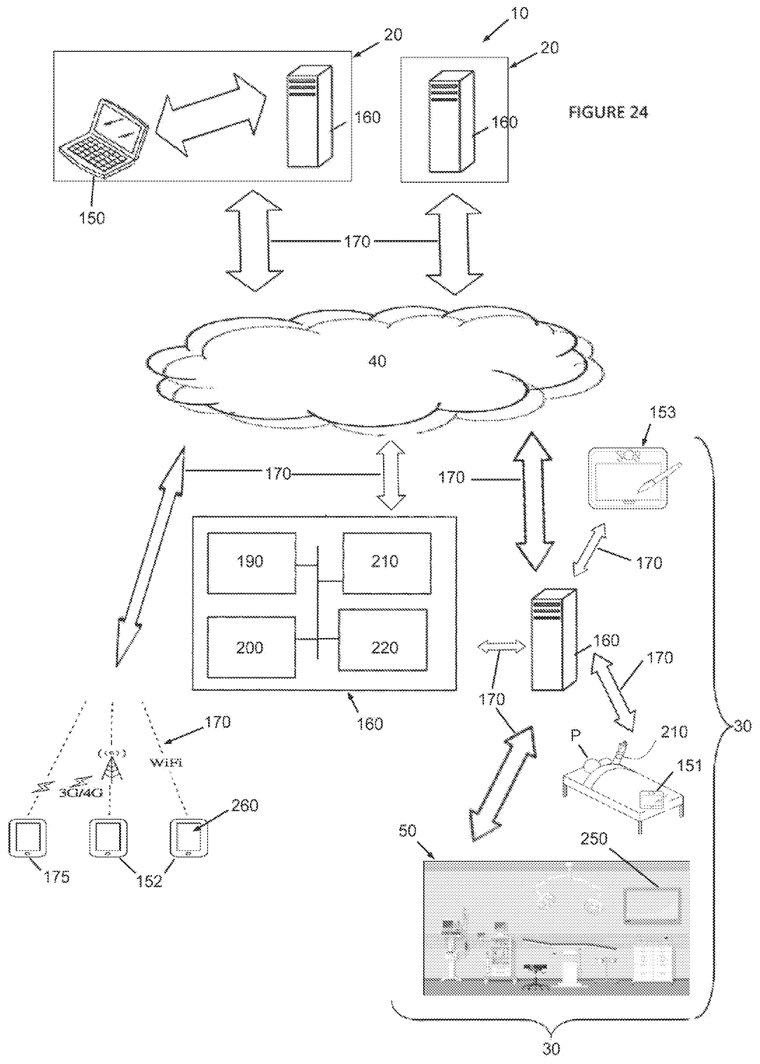

As shown in FIG. 24, the system 10 initially receives patient information data, electronic medical record information, either from another medical entity or doctor's office PO, or using an input device 150 at the physician's office 20, having sample screens as shown in FIGS. 1 and 2. As previously mentioned, the computer system 10 may comprise one or more local or remote computer servers 160 operable to transmit and process data and communicate via wired and wireless communication links and computer networks, generally referenced at arrows 170. As seen in FIGS. 1 and 2, the information data may include last name, first name, middle initial, patient status (current, completed, etc.), date of birth, date of surgery, surgeon, procedure planned, eye undergoing surgery (right or left), lens type to be used, what strength the eye is to be set for (near or distance vision), whether the Alcon® LenSx® or other laser equipment is planned to be used, whether an ocular response analyzer (ORA) is planned to be used, what specific post-operative medications are planned to be used, specific IOL measurements planned for lens and power, and the patient verification status. Additional data such as allergy information, any adverse medical reactions and prior surgical history may also be received. As shown in FIG. 3, planned surgical data information between the doctor and patient is also received into and stored within the system 10. The system may store the received data in a database or cloud-based store 40 for retrieving for processing. The data may be encrypted or otherwise secured using suitable protocols now known or later developed.

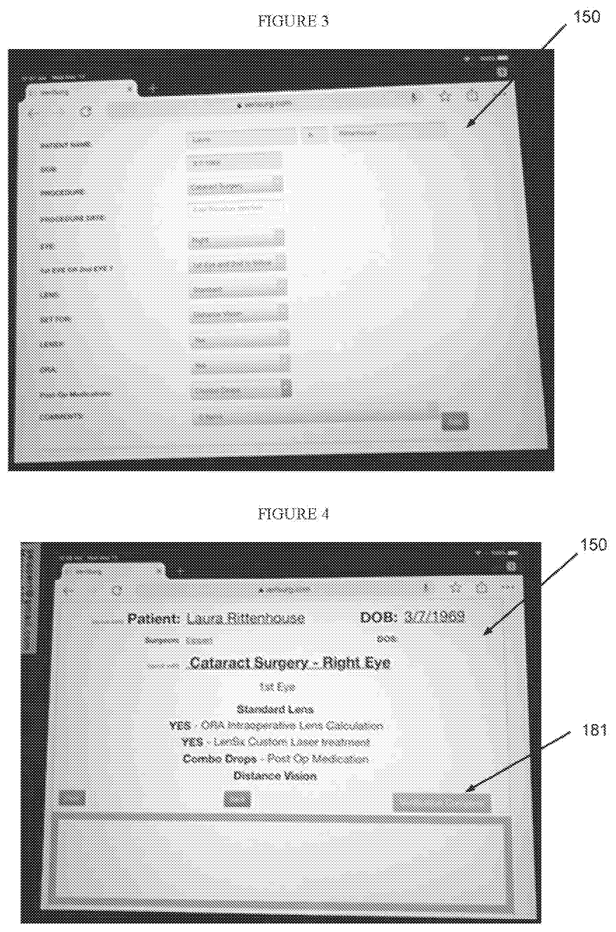

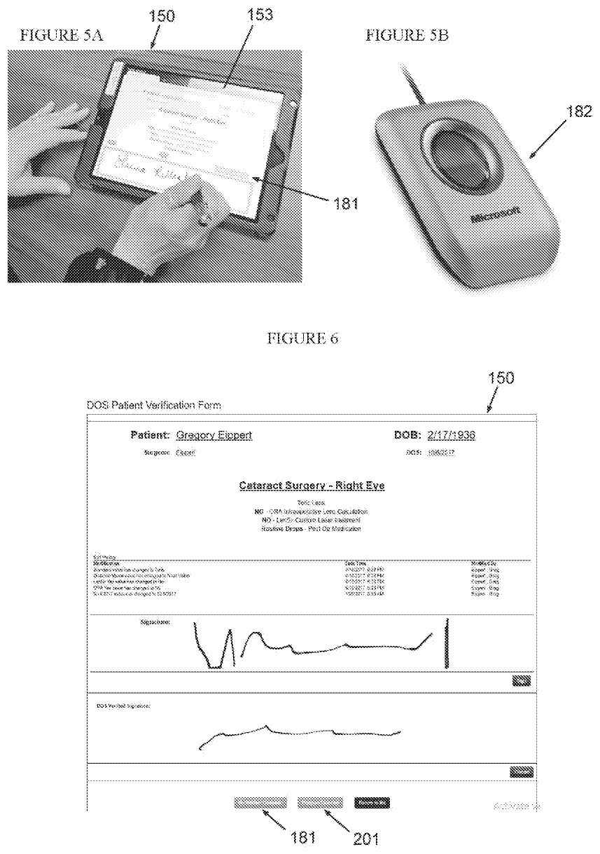

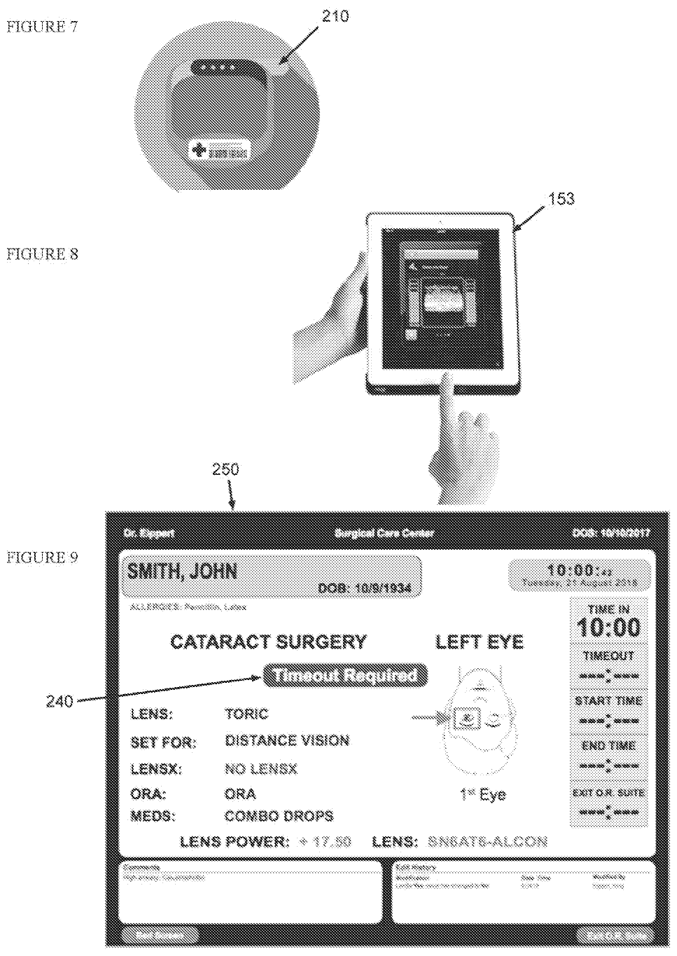

Once the patient reviews their information, as shown in FIG. 4 on a laptop, tablet or other information gathering device150, they are requested to verify the accuracy of the information. Using a verification module 180 of the system software, the patient electronically signs on the information gathering device 150, or tablet 153, as shown in FIG. 5A or an interconnected biometric device confirmation as shown in FIG. 5B. Once signed or confirmed, “clicking” or “pressing” the verification complete “button” 181 shown in FIG. 4, the verification module 180 time stamps the verification date and time in a format as shown in FIG. 6, and transmits the information for storage in a memory storage area 40, where it is stored for later access.

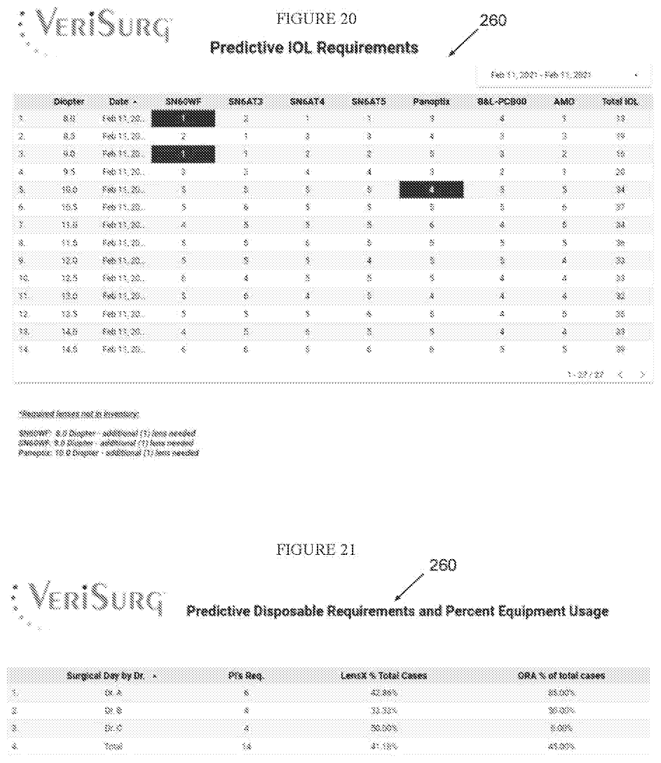

Authorized medical staff at surgery centers 30 using the system 10 are provided with alerts or reports 192, which may be manually or automatically generated by an inventory alert warning module 190 in advance of each surgical date, and on a real time basis, to ensure appropriate inventory for all implants, supplies and equipment are on hand and in place at the desired surgery center location. Authorized implant vendors may likewise be automatically or manually provided with an alert notification of inventory requirements, so that ordering can be done well in advance, or online by medical staff. Such alerts or reports 192 may be presented via e-mail, text message, multimedia message, voice message, a web page, etc. delivered to a number of suitable electronic or portable computing devices 175, as shown in FIG. 24. The inventory alert warning module 190 automatically presents the alerts or reports 192 to the medical staff. As shown in FIG. 20, the predictive number of IOL implants—by type, brand and diopter strength—which are required to be added to inventory are shown in the alerts provided. The medical staff may immediately coordinate ordering of the suggested IOL's missing from inventory, or such ordering may be automatically authorized by the system.

To calculate the necessary inventory and equipment requirements at a particular surgery center 30, the medical staff scans or otherwise electronically enters bar codes of all implants and equipment received at the surgery center and entered into inventory. Information regarding such on-hand implant inventory and equipment supplies is received into and stored within the system. As data regarding IOL and equipment needs for future surgical plans, historical physician usage data, as well as real time usage during surgical procedures, is entered and stored within the system, the predictive inventory alert warning module 190 within the system monitors, compares and calculates requirements for IOL implants and equipment, and alert warnings or reports as in FIG. 20 are generated as to whether additional inventory items are required, and when ordering is needed. The module generates and transmits reports to push the necessary alert warnings to the appropriate medical staff, and thereafter ordering may be either automatically or manually initiated. Alternatively, the appropriate medical staff may pull the alerts or reports using the predictive inventory alert warning module as desired to manually monitor and initiate necessary equipment and IOL ordering. FIG. 20 shows the needed IOL inventory highlighted in red within an inventory chart, as well as summarized in tables at the bottom. Extraction of IOL implant and equipment data from stored data sources in real time or batches, either directly or through the internet, is accomplished using various technologies and protocols. In real time, the data is preferably pre-processed to a structured data in a desired standardized format for processing.

On surgery day, the patient P arrives at a surgical center 30 and the

arrival time is documented and stored within the system 10. The system is then used via an information gathering device 150 to obtain stored patient identification information, which is retrieved for review by the patient using a re-verification module 200 of the system. As shown in FIG. 6, the patient's initial signature appears, and the patient is asked to re-confirm that the information is accurate by again electronically signing or providing finger print confirmation, as shown in FIG. 8. Any necessary changes or corrections may be made and re-verified and stored within the re-verification module 200. In the event the re-verification module 200 detects that a second confirmation signature by the patient P has not been provided, the system 10 provides an alert or report indicating that the surgery should not proceed. A verification complete button 181 is also provided as shown in FIG. 6.

The patient's RFID bracelet 210, as shown in FIG. 7, is then created using the “program bracelet” button 201 shown in FIG. 6. The bracelet 210 is placed on the patient's wrist or ankle, also seen in FIG. 24. To create the RFID chip for the bracelet 210, the patient's electronic information is retrieved from the system 10, and the bracelet 210 is read/scanned at various stages and locations during the patient's surgical experience. The first retrieval of the patient information using the RFID bracelet 210 occurs upon arrival to the pre-operative area of the surgery center, which time stamp is saved within the system. As previously discussed, beacon technology may be incorporated to provide automatic time stamping of the patient's movements throughout the surgery center 30. Additionally, such information may be provided to a bed monitor device 151, or together with a beacon positioning system, as the patient is transported past beacon devices located at various locations throughout the surgery centers.

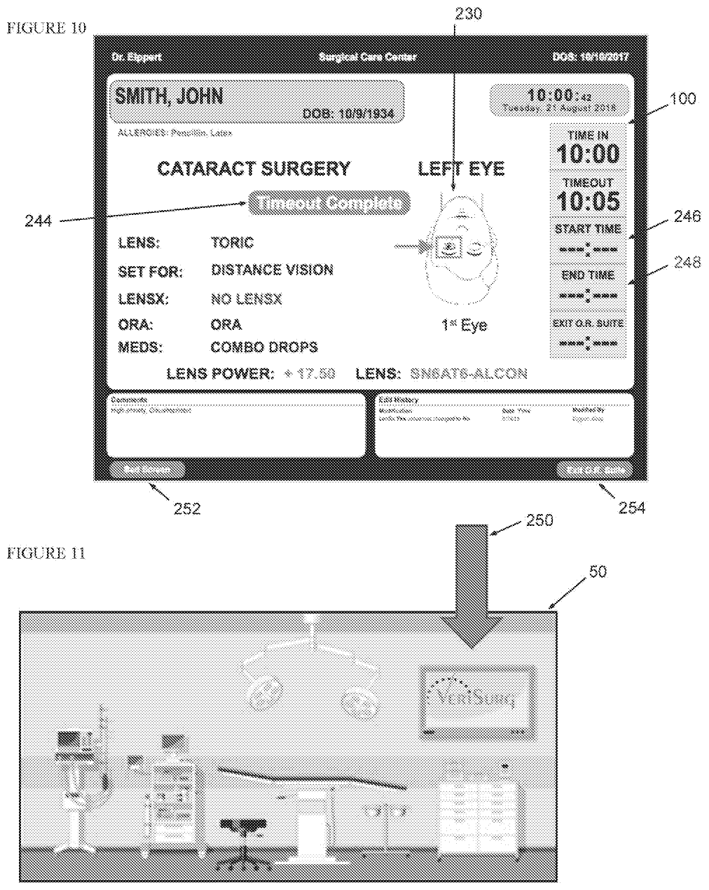

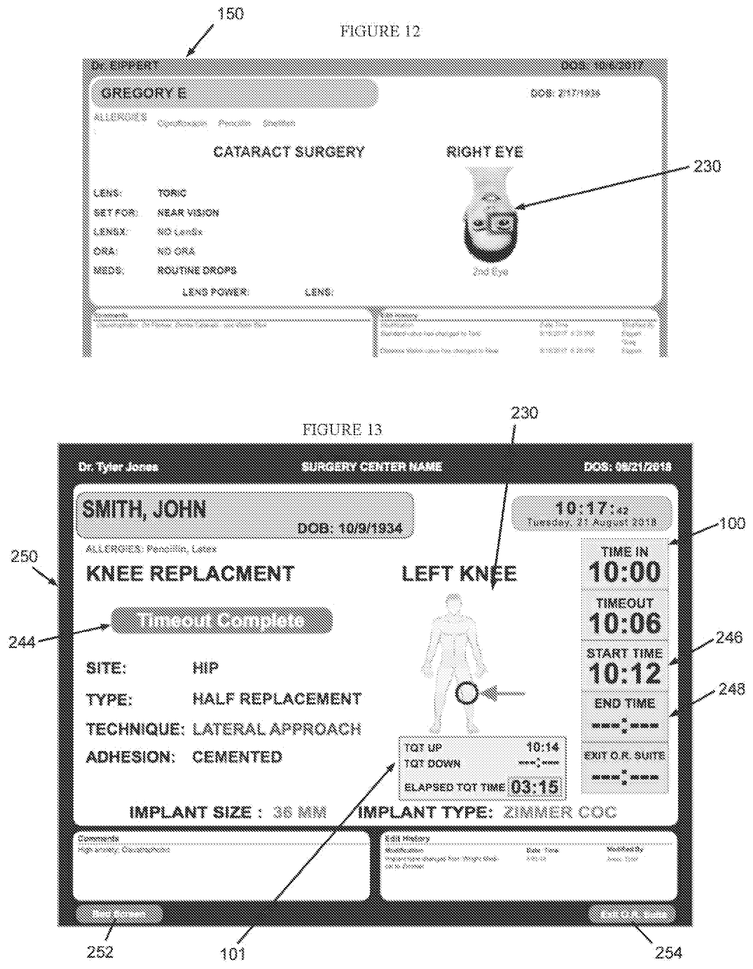

Arrival in the surgical suite or operating room 50, initiates the use of the time-out alert warning module 220 and another time stamp is stored within the system 10. The patient's ID bracelet 210 may be scanned, and the patient's information may be visually presented on an operating room computer and/or a large, for example 40 inch, “heads up” monitor 250 visible throughout the entire surgical suite 50, as in FIGS. 11 and 24. On screen, a visual image of a human form 230 with the specific location of the patient's surgery site will be highlighted, as in FIGS. 10, 12 and 13.

The system 10 displays the time-out alert warning 240 on screen until the time-out procedure is performed by the physician in the operating room 50 immediately before the planned procedure is initiated. As shown in FIG. 9, the time-out alert warning module 220 provides the time-out alert warning 240 in red on the operating room monitor 250, and pauses the system 10 until the proper confirmation of the physician's time-out review is received. Activation by the physician of an electronic confirmation “button” 240 in the time-out alert warning module 220, as shown in FIG. 9, is required to confirm the time-out procedure has been performed, and permits removal of the time-out alert warning to the green condition 244 shown in FIG. 10, as well as continued viewing and use of the system. The start and stop times 246, 248 taken to perform various procedures or transport times, as shown in FIG. 10, are stored for later historical data analysis. In the event the physician does not verify performance of the time out procedure, the surgery does not proceed.

Two additional operational features are noted in the sample system computer screens shown in FIG. 10. First, the “Bed Screen” button 252 in the bottom left hand corner may be used to transfer the displayed patient information from the OR system screen to the bed monitor device 151 screen. Second, the “Exit O.R. Suite” button 254 may be used to time stamp conclusion of the surgical procedure.

Following surgery, the patient moves to the post-operative area for recovery prior to discharge, and the bed monitor 151 travels with the patient P to document departure and arrival times at further locations. Prior to discharge, electronic information on the patient's ID bracelet 210 is permanently erased. Additionally, the inventory alert warning module 190 is updated with data regarding implant and equipment usage, and the system is used to again provide medical staff and any authorized vendors with an alert or report 260, regarding any updated inventory requirements, as previously discussed. FIGS. 18, 19 and 21 illustrate sample reports regarding an individual physician and their historical planned usage and percent usage of particular IOL's and equipment usage.

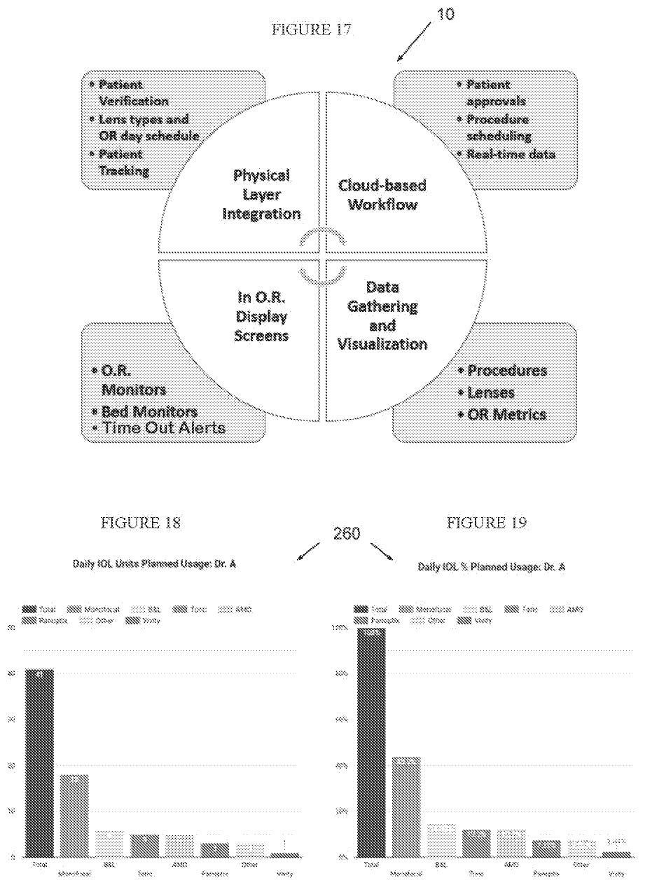

The system is preferably a cloud-based system with secure SSL authentication and encryption access which provides at least each of the features shown in FIGS. 17 and 24. The system is implemented using computer information gathering devices 150, 151, and mobile devices 152, 175, and includes multiple programmed processors, such as a personal computer 150, bed monitor 151, tablet 153, workstation, or servers 160. The system 10 may be accessed by communication connections to servers 160 and other data storage 40 through an internet, local area network (LAN), wide area network (WAN) or WiFi wireless connection. In this embodiment, both the computer or mobile devices and server run software that, when executed, enable the user to input instructions and information on the computer or mobile devices 150, 151, 152, auto-time stamp inputs and the starting and stopping of various procedures to document time spent on various tasks, document desired surgery room 50 set-up, equipment needs and inventory supply needs, to enable improved efficiencies in surgery suite preparation and inventory controls. The software sends the inputs to the server for conversion and output, and then electronically communicates, through the Internet, LAN, WAN or other wireless connection, to display the output on a display monitor, or print the output using a printer connected to the computer or mobile device.

As described, the system includes software that is run by a central processing unit of the system. The system may suitably include a number of standard input and output devices, including laptops, tablets, keyboard, mouse, touch screen, CD-ROM/CD-RW/DVD drive, disk drive or solid state drive, monitor and printer. For patient verifications and re-verifications, and patient bed monitors, the system is preferably provided with commercially available tablets 150, 151 having a 9 inch minimum screen size, for example, tablets such as an Android 11 using iOS14. The devices may also have a USB connection, or allow external hard drives, flash drives, chip readers, scanners, biometric device readers, such as finger print scanners, beacon technology, voice activated command software, and other programs and devices to enable input/output connection to the computers or mobile devices utilizing the system. All input/output devices, with the exception of the large heads up display screen 250 in the surgery suite 50 shown at the arrow in FIG. 11, require internet connections with preferably at least 10 MB per second synchronous speed. Native applications are preferred to accommodate both Windows® and Macintosh® based devices, with appropriate application programming interfaces to communicate between various integrated programs and electronic medical records.

It will be appreciated in light of the present description that the system 10 may be practiced in any of a number of different computing environments without departing from the spirit of the invention. For example, the system may be implemented in a network configuration with individual workstations connected to servers 160. Also, other input and output devices may be used as desired. For example, a remote user could access the server with a desk top computer, a laptop 150 utilizing the Internet or with a wireless handheld device such as cell phones 152, tablet 153s, and e-readers, such as iPad™ or iPod™ or the like. Additionally, patient location and time stamp information may be obtained using commercially available beacon technology (not shown) to interface with the system and provide real time automated data inputs to the system 10.

One embodiment of the invention has been designed for use on a personal computer running in Windows, preferably using Microsoft .NET framework. Another embodiment of the invention has been designed to run on a Linux-based server system. As described, it is contemplated that the system running on the computers and mobile devices will be operated by users in a medical care provider's office 20, administrative offices, surgery centers 30, out-patient care facility, hospital, nursing home or home setting, and is scalable to accommodate such multi-tenant architecture. The system preferably makes use of the cloud-based user interface for authorized medical staff to access reporting, configuration elements, potential system branding features, inventor adjustments and scanning. While the present system may be programmed or coded in any suitable programming language or programming environment such as Java, C++, Excel, Matlab, Python, etc, the preferred user interface is accessed via an Internet browser used on desktops, tablets and laptops, and further preferably uses a general purpose web scripting language, such as PHP.net, with an open source type relational database management programs, such as MySQL or MariaDB, for managing database information related to physician and surgery center analytics, as well as equipment and inventory control and reporting. As described, the inputs suitably include patient information and

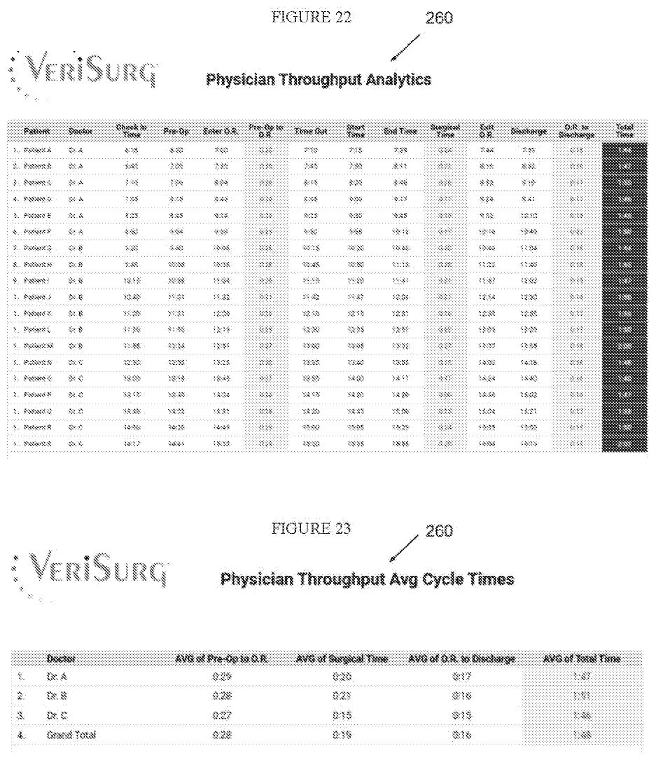

demographics, patient preferences, doctor preferences and constraints, facility equipment and inventory supplies, equipment and implant vendor information, across multiple facility locations, multiple medical care providers, and the like. The output information may appear on a display screen of the monitor or may also be printed out from a printer, or automatically provided to appropriate medical staff via interconnected electronic hand held devices for review and handling. A variety of output reports may be generated by the system for the medical staff in order to identify potential problems and improve efficiencies, such as the total patient time spent at a facility; the surgical times of specific medical staff, the time spent by a patient in room prior to a procedure start time, as well as total patient procedure times. The output information may also make use of other outputs as desired, for example, time stamped information may be used to satisfy various state and federal regulatory requirements. Samples of individual medical staff doctor reports are illustrated in FIGS. 14, with additional analytics shown in FIGS. 22 and 23.

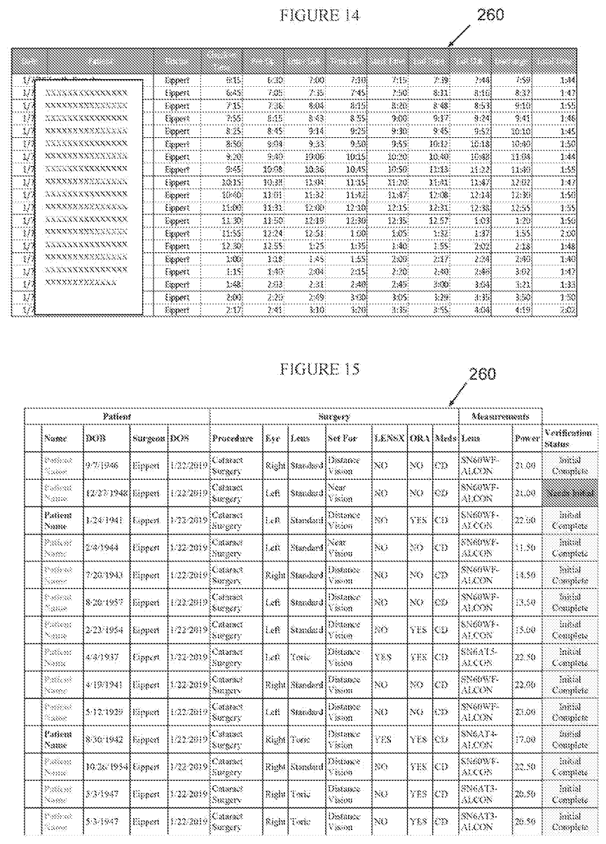

Additionally, a sample of a single surgery day report for an individual doctor is also shown in FIG. 15. The daily report includes patient specific information and planned implant information. Where changes to an original plan agreed upon by the doctor and patient are made, the system requirement for further verification is highlighted as requiring additional action.

While the present system has been disclosed in the context of various aspects of preferred embodiments, it will be recognized that the system may be suitably applied to other environments consistent with the following claims.

We claim:

1. A computer-implemented method for inputting patient information for specified surgical procedures, generating a time-out alert warning prior to such specified surgical procedures, and outputting patient information for specified surgical procedures comprising:
electronically receiving and storing by a programmed computer a set of information and dates for a specific patient surgery or medical procedure;
electronically receiving and storing by a programmed computer a patient verification of the set of information and dates stored regarding the specific patient surgery or medical procedure;
for the set of information stored regarding for the specific patient surgery or medical procedure, generating a required re-verification confirmation regarding the stored set of information prior to proceeding with the specific patient surgery or medical procedure;
for the set of information stored regarding the specific patient surgery or medical procedure, generating a required time-out alert warning for the specific patient surgery or medical procedure; and
pausing the programmed computer-implemented method prior to proceeding with the specific patient surgery or medical procedure until electronically receiving and storing a required confirmation response to the required time-out alert warning prior to proceeding with the specific patient surgery or medical procedure; and
re-generating a display of the stored set of information regarding the specific patient surgery or medical procedure following electronically receiving and storing the required confirmation response to the required time-out alert warning . . .

2. The computer-implemented method of claim 1, wherein the step of generating the required time-out alert warning includes generating a display of a human form having the location or site of the specific patient surgery or medical procedure to be performed highlighted for ease of visualization and confirmation of the correct location or site.

3. The computer-implemented method of claim 1, additionally including the steps of: monitoring an existing inventory of implants prior to the specific patient surgery or medical procedure,
calculating predicted implant inventory needs for implants prior to the specific patient surgery or medical procedure; and
generating and transmitting an inventory alert warning in a message format regarding predicted implant inventory needs for ordering implants.

4. A procedural and surgical information alert system, comprising:
a data store configured to receive and store data associated with a plurality of patients including planned surgical procedure information and planned IOL implants for a plurality of surgery centers;
a predictive module to monitor an existing inventory of IOL implants for a plurality of surgery centers, historical data of IOL implant usage at a plurality of surgery centers, and real time data for planned surgical procedure information using IOL implants, and calculate predicted IOL implant inventory needs for ordering IOL implants;
an inventory alert warning generated by the predictive module to transmit notification and information regarding predicted IOL implant inventory needs for ordering IOL implants to authorized surgery center staff and authorized IOL implant vendors in a message format;
the data store configured to electronically receive and store a patient verification of planned surgical procedure information and planned IOL implants; and
the data store configured to electronically receive and store a second patient verification requirement immediately prior to the planned surgical procedure and planned IOL implants.

5. The procedural and surgical information alert system, further comprising a time-out alert warning generated for the set of information stored regarding a physician confirmation of a time-out procedure performed immediately prior to a planned surgical procedure.

6. A computer-implemented method for generating procedure and surgical alert warnings for surgical procedures, comprising:
electronically receiving and storing by a programmed computer a set of information, dates and IOL implants needed for a specific patient surgery;
electronically receiving and storing by a programmed computer a patient verification of the set of information, dates and IOL implants needed regarding the specific patient surgery;
for the set of information stored regarding the specific patient surgery, generating a required patient re-verification confirmation regarding the stored set of information, dates and IOL implants prior to proceeding with the specific patient surgery;
for the stored set of information, dates and IOL implants regarding the specific patient surgery, generating a required time-out alert warning for the specific patient surgery;
pausing the computer-implemented method prior to proceeding with the specific patient surgery until electronically receiving and storing a required confirmation response to the required time-out alert warning prior to proceeding with the specific patient surgery;
re-generating a display of the stored set of information regarding the specific patient surgery following electronically receiving and storing the required confirmation response to the required time-out alert warning . . .
receiving and storing data associated with a plurality of patients with a planned specific patient surgery and planned IOL implants for a plurality of surgery centers;
monitoring an existing inventory of IOL implants for the plurality of surgery centers, historical data of IOL implant usage at the plurality of surgery centers, and real time data for the stored set of IOL implants;
calculating predicted IOL implant inventory needs for ordering IOL implants for a plurality of surgery centers; and
generating and transmitting an inventory alert warning to authorized surgery center staff and authorized IOL implant vendors in a message format regarding predicted IOL implant inventory needs for ordering IOL implants.

7. The computer-implemented method of claim 6, wherein the step of generating a display of the stored set of information regarding the specific patient surgery following the step of pausing the programmed computer method, includes generating a display of a human form having the location or site of the specific patient surgery to be performed highlighted for case of visualization and confirmation of the correct location or site.
